(19) United States
(12) Patent Application Publication
(10) Pub. No.: US 2009/0082775 A1
(43) Pub. Date: Mar. 26, 2009

(54) Spondylolisthesis reduction system and method

(76) Inventors: Moti Altarac, Irvine, CA (US); Joey Camia Reglos, Lake Forest, CA (US); Stanley Kyle Hayes, Mission Viejo, CA (US)

Correspondence Address: RIMAS LUKAS;VERTIFLEX, INC., 1351 CALLE AVANZADO, SAN CLEMENTE, CA, 92673, US

(21) Appl. No.: 12/329,423

(22) Filed: Dec. 5, 2008

Related U.S. Application Data
(63) Continuation-in-part of application No. 11/586,849, filed on Oct. 25, 2006.
Application No. 11/586,849 is a continuation-in-part of application No. 12/077,462, filed on Mar. 19, 2008.
(60) Provisional application No. 61/005,611, filed on Dec. 6, 2007.
(60) Provisional application No. 60/919,198, filed on Mar. 20, 2007.

(51) Int. Cl. A61B 17/58; A61B 17/70; A61B 17/04; A61B 19/00

(52) U.S. Cl. 606/90; 606/246; 606/301; 606/308; 128/898

(57) ABSTRACT

A percutaneous spondylolisthesis reduction instrument and method are disclosed for minimally invasive surgery. The spondylolisthesis reduction instrument includes tangs threadingly connected to an upper knob at the proximal end and configured to connect with a tower construct at the distal end. When connected to a tower which is attached to a bone fastener assembly implanted in a vertebral body, rotation of the upper knob retracts the tangs and the connected tower construct and vertebral body to reduce spondylolisthesis.

### CROSS-REFERENCE TO RELATED APPLICATIONS

This application claims the benefit of and is a continuation-in-part of U.S. Provisional Patent Application Ser. No. 61/005,611 entitled “Spondylolisthesis reduction system and method” filed on Dec. 6, 2007 which is incorporated herein by reference in its entirety. This application is also a continuation-in-part of co-pending U.S. patent application Ser. No. 11/586,849 entitled “Systems and methods for stabilization of bone structures” filed on Oct. 25, 2006 incorporated herein by reference in its entirety. This application is also a continuation-in-part of co-pending U.S. patent application Ser. No. 12/077,462 entitled “Rod reducer” filed on Mar. 19, 2008 incorporated herein by reference in its entirety which is a non-provisional patent application of U.S. Provisional Patent Application Ser. No. 60/919,198 entitled “Rod reducer” filed on Mar. 20, 2007 incorporated herein by reference in its entirety.

### FIELD

The present invention generally relates to medical devices for the spine. In particular, the present invention relates to instruments and methods for correcting spondylolisthesis.

### BACKGROUND

Spondylolisthesis, known as “spondy”, is a displacement disorder of the lumbar or cervical spine, in which one vertebral body is forwardly displaced over another vertebral body as shown in FIG. 1. Spondylolisthesis may be caused by a traumatic event or by degeneration of the spine. At times, the displacement disorder is accompanied by or caused by a fracture or partial collapse of one or more vertebrae or degeneration of a disc in the spine. Patients who suffer from such conditions can experience moderate to severe distortion of the thoracic skeletal structure, diminished ability to bear loads, loss of mobility, extreme and debilitating pain, and oftentimes suffer neurological deficits in nerve function.

Spinal correction systems may be used in orthopedic surgery to correct a deformity or misalignment caused by spondylolisthesis, as well as to stabilize and/or fix vertebral bodies in a desired relationship relative to each other. A standard surgical procedure for correcting spondylolisthesis involves first distracting the vertebrae at the level that the spondylolisthesis occurs, pulling the forward-translated vertebra back into alignment with the rest of the spinal column, and then stabilizing the spine while the vertebrae are held in the aligned position using spinal implants consisting of anchoring devices and rigid or semi-rigid spinal fixation elements. An interbody fusion device may also be used to give further stability and correction of the disc height, which may be compromised during the spondylolisthesis event. Compression across the vertebrae may be applied across the construct to set the correct balance of forces in the region.

The spinal fixation element used in such spinal correction systems is generally a relatively rigid fixation rod or plate that is coupled to a bone by attaching the spinal fixation element to various anchoring devices, such as hooks, bolts, wires or screws. The spinal fixation element can extend between two bone regions to effect stabilization, positioning, reduction or fixation of the bones. The spinal fixation element can extend between two bone regions to effect stabilization, positioning, reduction or fixation of the bones. The spine fixation element can have a predetermined contour that has been designed according to the properties of the target implantation site and, once installed, the spinal fixation element holds the bones in a desired spatial relationship, either until desired healing or spinal fusion has occurred, or for some longer period of time.

Prior surgical procedures and devices for correcting spondylolisthesis are inadequate and present several difficulties. For example, the technique of pulling the forwardly displaced vertebral body back into alignment before attaching the spinal fixation elements can be difficult. For example, the forces required to pull the vertebral body back into alignment can be very large and/or uneven, difficult to control and/or cause damage to the patient and/or implants. In addition, significant force is required to hold the vertebral body in alignment during subsequent attachment of the spinal fixation elements. Specialized and improved instruments are required to carry out the procedure, in particular, to treat spondylolisthesis via a minimally invasive, percutaneous approach to the spine.

### SUMMARY

According to one aspect of the invention, a spondylolisthesis reduction system for a patient's spine is provided. The system includes a bone anchor implantable into a vertebral body of the patient's spine. The bone anchor has an upper rod receiving portion connected to a lower shank portion. The system also includes a guide tube having an upper portion and a lower portion with the lower portion being connectable to the bone anchor. The guide tube also has a longitudinal opening. And the system further includes an instrument connectable to the upper portion of the guide tube and configured to move the lower portion of the guide tube relative to the upper portion.

According to another aspect of the invention, a spondylolisthesis reduction system for a patient's spine is provided. The system includes an upper knob and at least one hook is connected to the upper knob and configured such that the at least one hook is capable of longitudinal translation relative to the upper knob. The at least one hook is further configured to be connectable with a tower having a longitudinal opening. The at least one hook is configured to longitudinally translate a connected tower along with the longitudinal translation of the at least one hook. The tower is configured to be connectable to a bone fastener assembly implanted in a vertebral body of the patient's spine. Translation of the at least one hook, connected tower and vertebral body relative to the upper knob reduces spondylolisthesis of the patient's spine. In another variation, the upper knob is configured to connect directly to an adjacent tower or to an upper portion of a tower that is separable from a lower portion of the tower.

According to another aspect of the invention a method for reducing spondylolisthesis in a patient's spine is provided. The method includes the step of providing at least a first bone anchor implanted in a first vertebral body. At least a second bone anchor is implanted in a second vertebral body. A first tower is provided and connected to one of the first bone anchors. A second tower is provided. The second tower has at least one longitudinal opening, an upper portion and a lower portion. The second tower is connected to one of the second bone anchors. The first tower is connected to the upper portion of the second tower. An instrument comprising an upper portion connected to at least one prong is provided. The instrument is inserted into the second tower. The at least one prong is connected to the second tower. The prong is moved proximally to move the second tower, the connected second bone anchor, and the connected second vertebral body proximally to align the first and second vertebral bodies.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates a side view of a portion of a human spine with one vertebral body displaced over another in the direction of the arrows.

FIG. 2a illustrates a perspective view of a spondy reduction instrument according to the present invention.

FIG. 2b illustrates a side cross-sectional view of a spondy reduction instrument according to the present invention.

FIG. 3 illustrates cross-sectional and perspective views of an upper knob of a spondy reduction instrument according to the present invention.

FIG. 4 illustrates cross-sectional and perspective views of tangs of a spondy reduction instrument according to the present invention.

FIG. 5 illustrates cross-sectional and perspective views of an upper cage of a spondy reduction instrument according to the present invention.

FIG. 6 illustrates a perspective view of a lower cage of a spondy reduction instrument according to the present invention.

FIG. 7 illustrates perspective and cross-sectional views of a lower knob of a spondy reduction instrument according to the present invention.

FIG. 8 illustrates cross-sectional and perspective views of a cage connecting pin of a spondy reduction instrument according to the present invention.

FIG. 9 illustrates a side view of an inner shaft of a spondy reduction instrument according to the present invention.

FIG. 10 illustrates perspective view of a spinal segment implanted with three bone fastener assemblies without caps.

FIG. 11a illustrates a perspective view of two alignment guides connected to two of four towers that are connected to four bone fastener assemblies implanted in a spinal segment.

FIG. 11b illustrates a side view of the assembly of FIG. 11a.

FIG. 12a illustrates a perspective view of a tower.

FIG. 12b illustrates a cross-sectional view of a tower.

FIG. 13a illustrates a perspective view of an alignment guide.

FIG. 13b illustrates a cross sectional view taken along line B-B of FIG. 13c of an alignment guide.

FIG. 13c illustrates a top view of an alignment guide.

FIG. 14a illustrates a perspective view of four alignment guides connected to four towers connected to four bone fastener assemblies implanted in a spinal segment.

FIG. 14b illustrates a side view of the assembly of FIG. 14a.

FIG. 15a illustrates a perspective view of four alignment guides connected to four towers connected to four bone fastener assemblies implanted in a spinal segment with two rods.

FIG. 15b illustrates a side view of the assembly of FIG. 15a.

FIG. 16a illustrates a perspective view of two spondy reduction instruments without inner shafts according to the present invention in juxtaposition with the assembly of FIG. 15a.

FIG. 16b illustrates a side view of the assembly of FIG. 16a.

FIG. 17a illustrates a perspective view of two spondy reduction instruments with inner shafts according to the present invention connected to two alignment guides.

FIG. 17b illustrates a side view of the assembly of FIG. 17a.

FIG. 18 illustrates a partial cross-sectional view of a spondy reduction instrument according to the present invention connected to a tower.

FIG. 19a illustrates a side view of a screw locking tool according to the present invention inserted into one of the alignment guides and towers and into the seat of the bone fastener assembly.

FIG. 19b illustrates a partial perspective view of the assembly of FIG. 19a.

FIG. 20a illustrates a perspective view of two locking tools inserted over the upper knob of two spondy reduction instruments according to the present invention connected to two of four alignment guides and towers connected to bone fastener assemblies implanted in a spinal segment.

FIG. 20b illustrates a side view of the assembly of FIG. 20a.

FIG. 21a illustrates a perspective view of two spondy reduction instruments according to the present invention connected to two of four alignment guides and towers connected to bone fastener assemblies implanted in a spinal segment of which the spondylolisthesis has been reduced.

FIG. 21b illustrates a side view of the assembly of FIG. 21a.

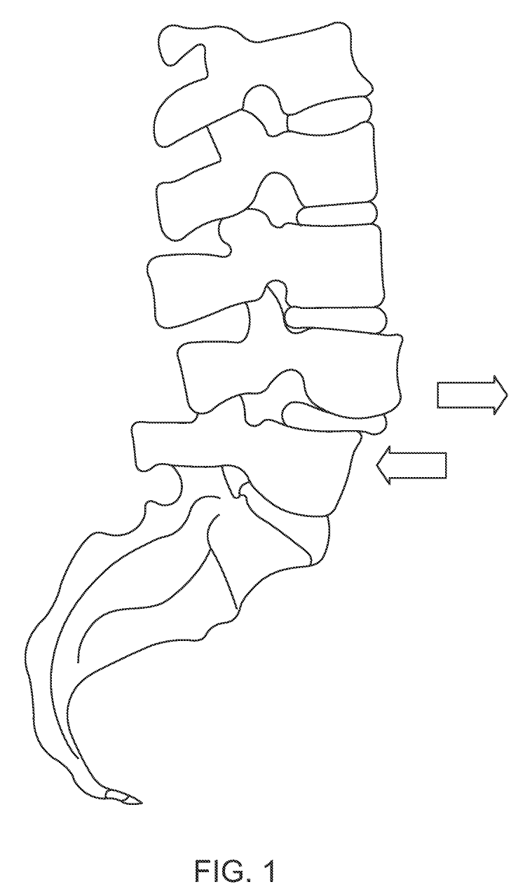

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device typically inserted inside a patient's body first. Thus, for example, the implant or instrument end first inserted inside the patient's body would be the distal end of the implant, while the implant or instrument end to last enter or remain outside the patient's body would be the proximal end of the implant or instrument.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention is described in the accompanying figures and text as understood by a person having ordinary skill in the field of spinal implants. Like numerals are used to describe like parts wherever possible.

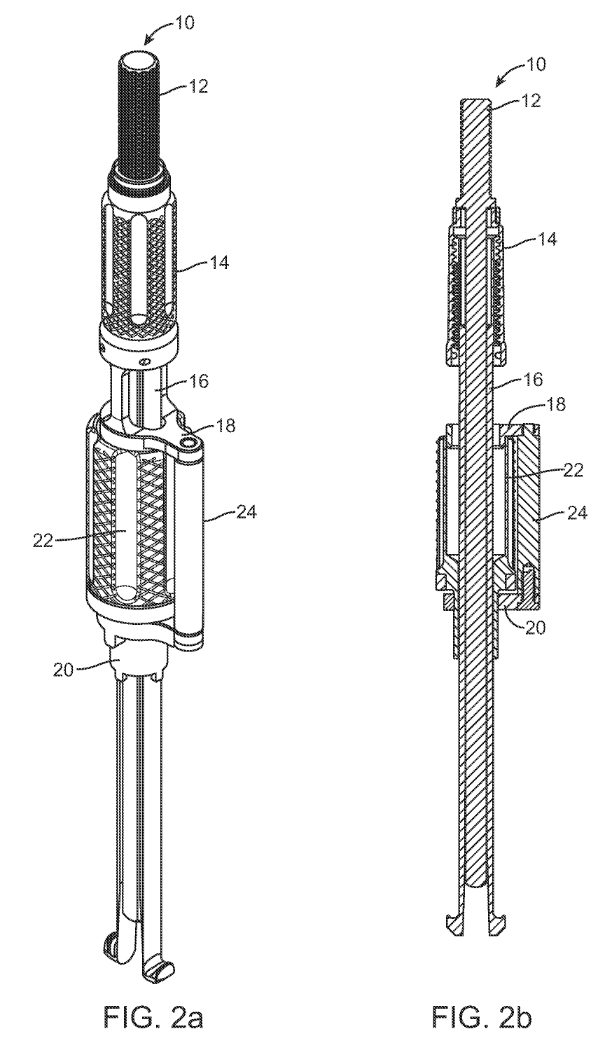

Turning now to FIGS. 2a and 2b, there is shown a spondy reduction instrument 10 according to the present invention. The spondy reduction instrument 10 includes an inner shaft 12, an upper knob 14, tangs 16, an upper cage 18, a lower cage 20, a lower knob 22, and a cage connecting pin 24.

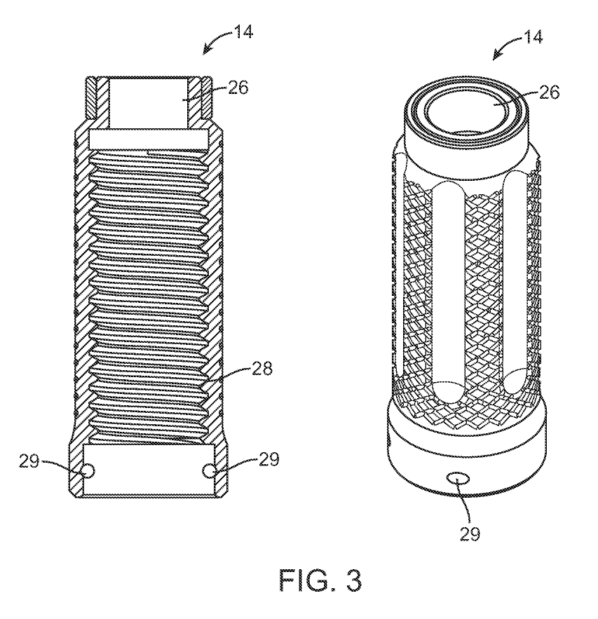

Turning now to FIG. 3, there is shown the upper knob 14 of the spondy reduction instrument 10. The upper knob 14 includes a central bore 26 having a threaded portion 28 between the proximal and distal ends. Pin holes 29 are formed at the distal end.

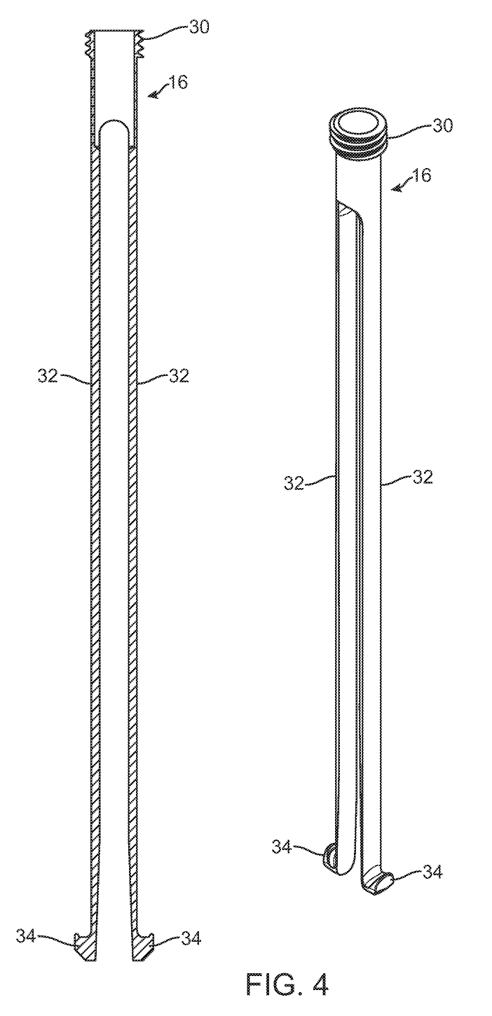

Turning now to FIG. 4, there is shown the tangs 16 of the spondy reduction instrument 10. The tangs 16 include a threaded portion 30 at the proximal end. The proximal end of the tangs 16 is configured to be inserted into the distal end of the upper knob 14 and the threaded portion 30 of the tangs 16 is sized to engage with the threaded portion 28 of the upper knob 14 and to connect therewith. The tangs 16 include a pair of distally extending prongs 32 having outwardly extending hooks 34 at the distal end. The prongs 32 are capable of being flexed inwardly and outwardly and constitute cantilevered flexible portions of the tangs 16.

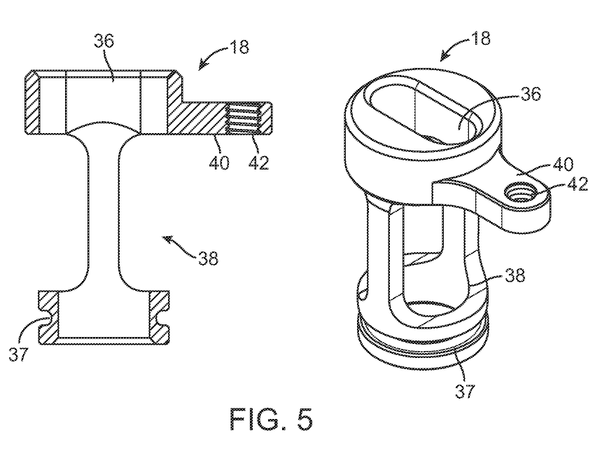

Turning now to FIG. 5, there is shown the upper cage 18 of the spondy reduction instrument 10. The upper cage 18 includes a longitudinally extending opening 36 and lateral cage windows 38. The proximal end of the upper cage 18 includes a circumferential retaining notch 37 and is configured to be inserted into the distal end of the upper knob 14. The distal end of the upper cage 18 is configured to be inserted into the proximal end of the lower knob 22. The longitudinal opening 36 is sized to receive the prongs 32 with the lateral cage windows 38 providing the user with finger access to the prongs 32 that are located inside the longitudinal opening 36 when the instrument is assembled. The upper cage 18 further includes an outwardly extending flange 40 with a threaded flange bore 42.

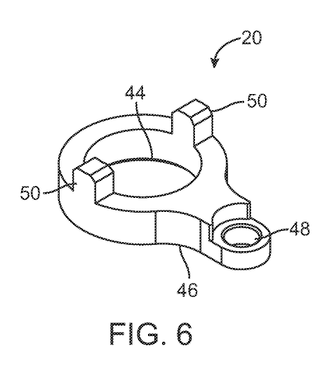

Turning now to FIG. 6, there is shown the lower cage 20 of the spondy reduction instrument 10. The lower cage 20 includes a central bore 44, an outwardly extending flange 46 with a flange bore 48. Two locator extensions 50 extend distally from the lower cage 20.

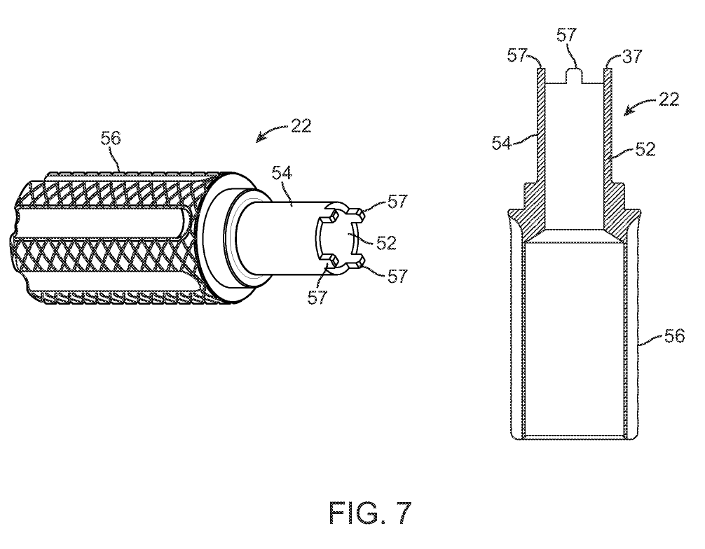

Turning now to FIG. 7, there is shown the lower knob 22 of the spondy reduction instrument 10. The lower knob 22 includes a longitudinal central bore 52, a distal portion 54 and a proximal portion 56. The distal portion 54 has a smaller outer diameter relative to the proximal portion 56 and at least one extension 57. The smaller distal portion 54 is sized to be inserted into the bore 44 of the lower cage 20 and the longitudinal central bore 52 is sized to receive the prongs 32 therein.

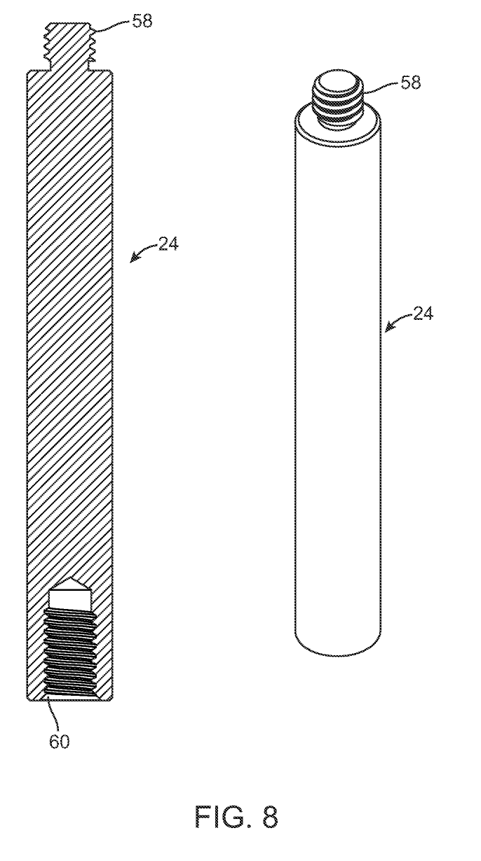

Turning now to FIG. 8, there is shown the cage connecting pin 24 of the spondy reduction instrument 10. The cage connecting pin 24 includes a threaded extension 58 at the proximal end configured to engage with the threaded flange bore 42 of the upper cage 18. At the distal end, the cage connecting pin 24 includes a threaded bore 60 configured to receive a threaded pin (not shown) that is passed through the flange bore 48 of the lower cage 20 for securement of the cage connecting pin 24 to the upper and lower cages 18, 20.

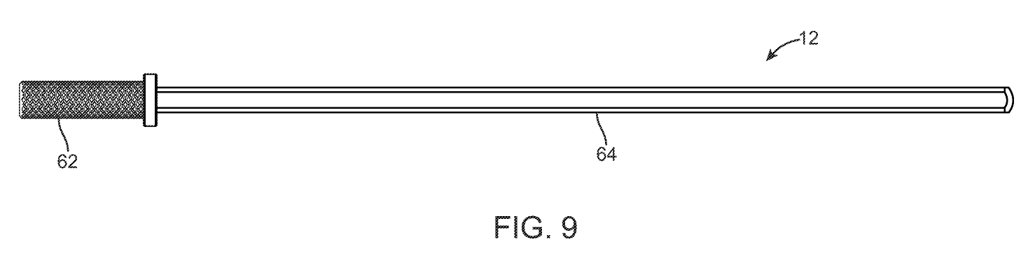

Turning now to FIG. 9, there is shown the inner shaft 12 of the spondy reduction instrument 10. The inner shaft 12 includes a proximal finger portion 62 and a shaft portion 64 configured and sized to spread apart the prongs 32 when the inner shaft 12 is inserted into the instrument.

The assembly of the spondy reduction instrument 10 will now be described with reference to FIGS. 2 through 9. The proximal threaded portion 30 of the tangs 16 is inserted into the distal end of the upper knob 14 and threaded to the threaded portion 28 of the central bore 26. The upper cage 18 is passed over the prongs 32 with the prongs 32 located inside the longitudinal opening 36 until the proximal end of the upper cage 18 is inserted into the distal end of the upper knob 14. Pins (not shown) are inserted into pin holes 29 of the upper knob 14 to engage with the circumferential notch 37 of the upper cage 18 such that the upper cage 18 is connected to the upper knob 14 and allowed to rotate with respect to it. The tangs 16 are captured within the threaded portion 28 of the upper knob 14 and allowed to travel longitudinally therein by a total distance of approximately 20 to 60 millimeters with longitudinal travel being distally limited by the inserted upper cage 18.

The distal portion 54 of the lower knob 22 is inserted into the central bore 44 of the lower cage 20. The distal end of the cage connecting pin 24 is inserted into the flange bore 48 and a threaded pin (not shown) is threaded into the threaded bore 60 to secure the cage connecting pin 24 to the lower cage 20. The distal end of the upper cage 18 is inserted into the proximal end of the lower knob 22 and the threaded extension of the cage connecting pin 24 is threaded into the threaded flange bore 42. Thereby, the lower knob 22 is connected or caged between the upper and lower cages 18, 20 such that it is permitted to rotate. The upper cage 18, lower cage 20 and cage connecting pin 24 form a bracket for the lower knob 22. The inner shaft 12 is inserted into the proximal end of the central bore 26 of the upper knob 14 such that the shaft portion 64 extends through the upper knob 14, upper cage 18, lower cage 20 and lower knob 22 and is located between the prongs 22. As the inner shaft 12 is inserted in between the prongs 22, the prongs 22 are spaced apart by the thickness of the shaft portion 64. In one variation, the shaft portion 64 is rectangular in shape such that insertion of the inner shaft 12 is directionally limited by the shape of the longitudinal opening 36 in the upper cage 18. Other variations include a square, round, elliptical or any other suitable cross-sectional shape.

The spondy reduction instrument 10 is designed for use with a system for implanting a spinal stabilization apparatus in a patient that is described in U.S. patent application Ser. No. 11/586,849 entitled “Systems and methods for stabilization of bone structures” filed on Oct. 25, 2006 incorporated herein by reference in its entirety and U.S. patent application Ser. No. 11/362,366 entitled “Systems and methods for stabilization of bone structures” filed on Feb. 23, 2006 incorporated herein by reference in its entirety. The spinal stabilization apparatus described in the aforementioned patent application includes at least a first bone fastener assembly and a second bone fastener assembly. Of course, other similar systems are employable with the current invention.

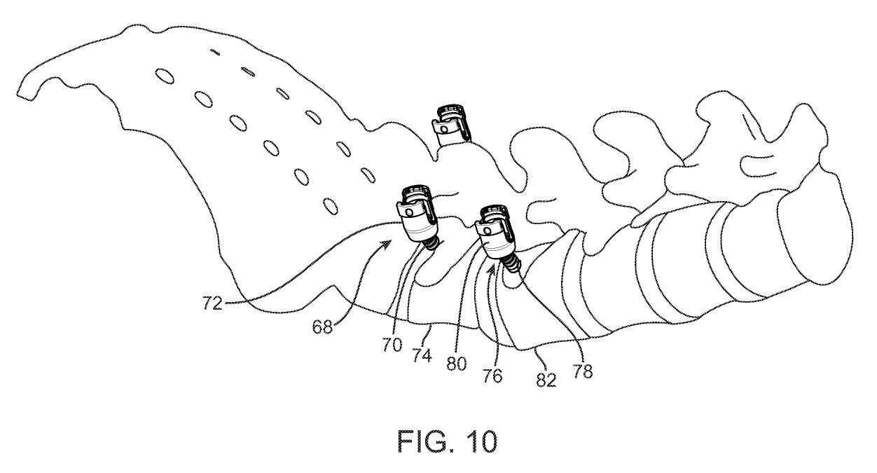

Turning now to FIG. 10, there is shown a spinal segment. The first bone fastener assembly 68 comprises a first bone screw 70 coupled to a first collar 72. The first bone fastener assembly is anchored into a first vertebral body 74 at a first target location. The second bone fastener assembly 76 comprises a second bone screw 78 coupled to a second collar 80. The second bone fastener assembly 76 is anchored into a second vertebral body 82 at a second target location wherein the second vertebral body 82 is a vertebral body adjacent to the first vertebral body 74. Typically, an elongate member or rod (not shown) is inserted and secured to the first collar 72 and to the second collar 80. This spinal stabilization apparatus is installed along one side of the vertebral bodies 74, 82 with the first and second screws 70, 78 typically being implanted in the pedicles on the same side of the vertebrae. Generally, a second spinal stabilization apparatus is installed along the other side of the spinous processes of the same vertebral bodies with third and fourth bone fastener assemblies comprising screws with collars being implanted in pedicles opposite from the first two and a second elongate member is secured to the third and fourth collars such that the elongate bodies run generally along the length of the spine. With the spinal stabilization apparatuses in place, adjacent vertebrae are supported and held apart in a relatively fixed position by the elongate members. Once the system has been assembled and fixed to a series of two or more vertebrae, it constitutes a rigid device substantially preventing the vertebrae from moving relative to one another. This rigidity enables the devices to support all or part of the stresses instead of the stresses being born by the series of damaged vertebra.

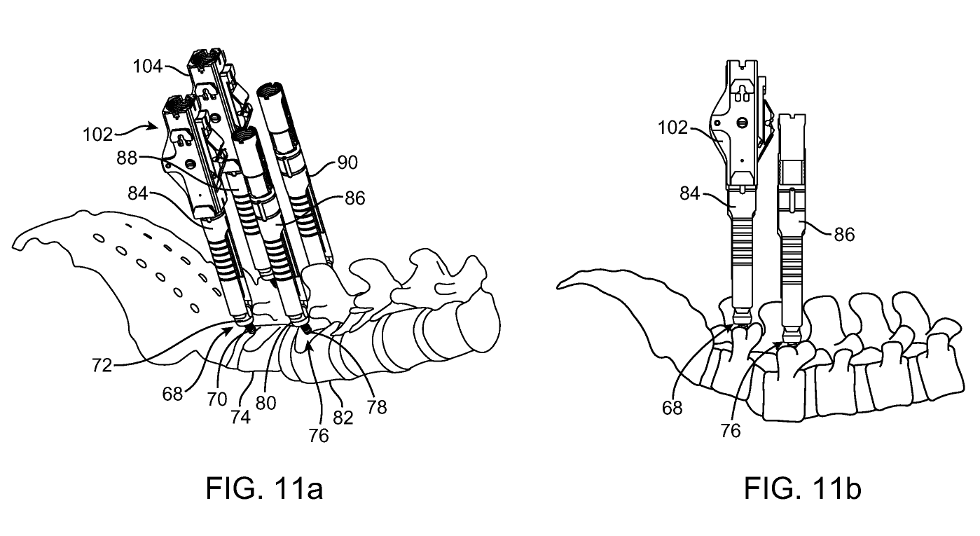

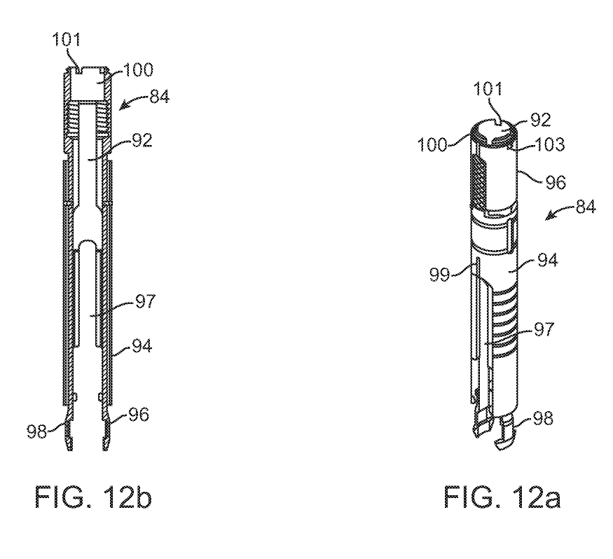

Turning now to FIGS. 11a and 11b, there is shown a spinal segment with a first vertebral body 74 adjacent to a second vertebral body 82 with first and second bone fastener assemblies 68, 76 installed. As shown, spondylolisthesis between the first and second vertebral bodies 74, 82 has resulted in the first vertebral body 74 shifting posteriorly relative to the second vertebral body 82. FIG. 11 also depicts a first tower 84 and a second tower 86 as well as a third and fourth tower 88, 90. A typical tower or guide tube construct is illustrated in FIGS. 12a and 12b.

Turning briefly now to FIGS. 12a and 12b, the typical tower such as the first tower 84 is generally cylindrical or tubular in shape having a longitudinal opening 92 extending between the proximal and distal ends. The tower 84 includes an outer sleeve 94 slidingly connected to an inner sleeve 96 and a lock 100 threadingly engaged with the threaded inner surface of the inner sleeve 96 at proximal end of the tower 84. The proximal end of the lock 100 includes notches 101 for engagement with a complementarily notched locking tool. The lock 100 is free to threadingly translate longitudinally in the inner threaded portion of the proximal end of the inner sleeve 96.

Still referencing FIGS. 12a and 12b, the proximal end of the inner sleeve 96 extends out from the proximal end of the outer sleeve 94. At the proximal end, the inner sleeve 96 includes at least one notch 103 (FIG. 12a) formed in the sidewall. At the distal end, the inner sleeve 96 includes a pair of collar engaging prongs 98 configured to correspond to the outer shape of a collar of a bone fastener assembly. With the collar engaging prongs 98 extended beyond the distal end of the outer sleeve 94, the distal end of the inner sleeve 96 is ready to receive the collar therein. With the lock 100 disposed inside the inner sleeve 96 at the proximal end, rotation of which pushes the outer sleeve 94 over the inner sleeve 94 retracting the prongs 98 into the outer sleeve 94 and deflecting the collar engaging prongs 98 slightly inwardly to lock onto the collar. In such a manner, the first and second towers 84, 86 are attached to the first and second collars 72, 80, respectively, and the third and fourth towers 88, 90 are attached to the third and fourth collars, respectively. Two channels 97 that are oppositely located from one another and extend longitudinally from the distal end toward the proximal end are formed in the sidewall of the outer sleeve 94. Also, at least one alignment marker 99 is provided on the outer sleeve 94 which is in line with at least one of the two channels 97.

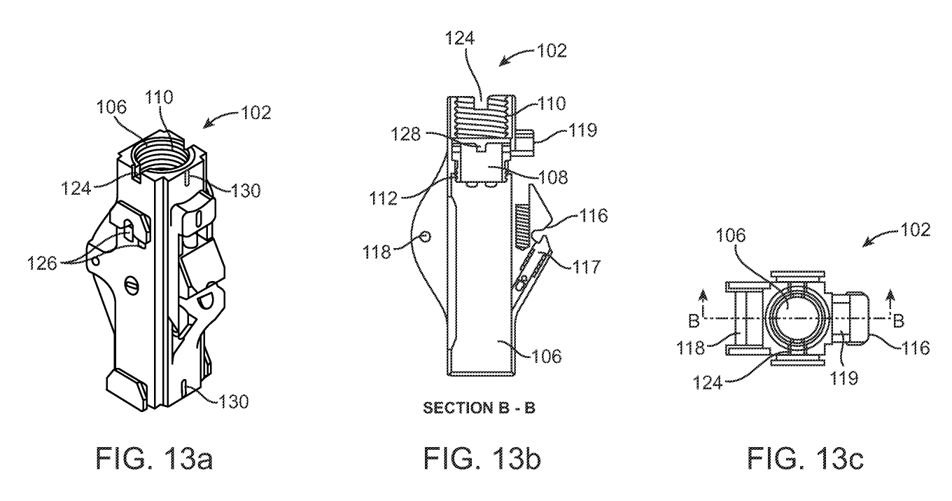

Referring back to FIGS. 11a and 11b, there is shown a first alignment guide 102 connected to the first tower 84 and a third alignment guide 104 connected to the third tower 88. The first and third alignment guides 102, 104 are identical and shown in greater detail in FIGS. 13a, 13b and 13c.

Turning briefly now to FIGS. 13a, 13b and 13c, the typical alignment guide 102 or upper portion of the tower has a longitudinal opening 106 extending between the proximal and distal ends. The alignment guide 102 includes an alignment lock 108 disposed inside the longitudinal opening 106 near the proximal end. The alignment lock 108 includes a threaded distal portion 112 and is captured inside the longitudinal opening 106. At least one notch 128 is formed in the sidewall of the proximal end of the alignment lock 108. The alignment guide 102 also includes a threaded insert 110 welded into the longitudinal opening 106. The alignment lock 108 is free to rotate within the longitudinal opening with longitudinal travel limited by the threaded insert 110 at the proximal end and a ledge (not shown) at the distal end.

Still referencing FIGS. 13a, 13b and 13c, the alignment guide 102 includes a hook 116 formed on the outside of the alignment guide 102 and a post 118 formed outside of the alignment guide opposite from the hook 116. The hook 116 is configured to hook onto the post 118 of an adjacently located alignment guide. The hook 116 includes a spring biased portion 117 that permits an adjacent post to snap therein from a substantially lateral direction. A hook release 119 is provided to push the spring biased hook 116 back to release an adjacent post. The alignment guide is configured to connect with the proximal end of a tower. The alignment guide 102 further includes at least one notch 124 in the sidewall at the proximal end of the alignment guide and at least one alignment marker 130 on the outer sidewall. At least one window 126 is formed in the sidewall of the alignment guide in a location proximate to the threaded distal portion 112 of the alignment lock 108 so that during percutaneous procedures the contact of the lock 108 with the tower is easily ascertained.

Turning back to FIGS. 11a and 11b, at least one of the first and third alignment guides 102, 104 is connected to either one or both of the first and third towers 84, 88. For example, the first alignment guide 102 or upper portion is placed over the first tower 84 or lower portion such that the proximal end of the first tower 84 is inserted into the longitudinal opening 106 of the first alignment guide 102. The alignment guide 102 is locked to the first tower 84 by rotation of the alignment lock 108. A locking tool with projections corresponding to the notches 128 (FIG. 13) in the alignment guide lock 108 is inserted into the proximal end of the alignment guide 102 until the projections contact the notches 128. The locking tool thusly engaged is then turned to rotate the alignment lock 108 which threads the threaded portion 112 of the lock 108 to the threaded inner surface of the inner sleeve 96 of the tower 84. The third alignment guide 104 is connected to the third tower 88 in the same manner.

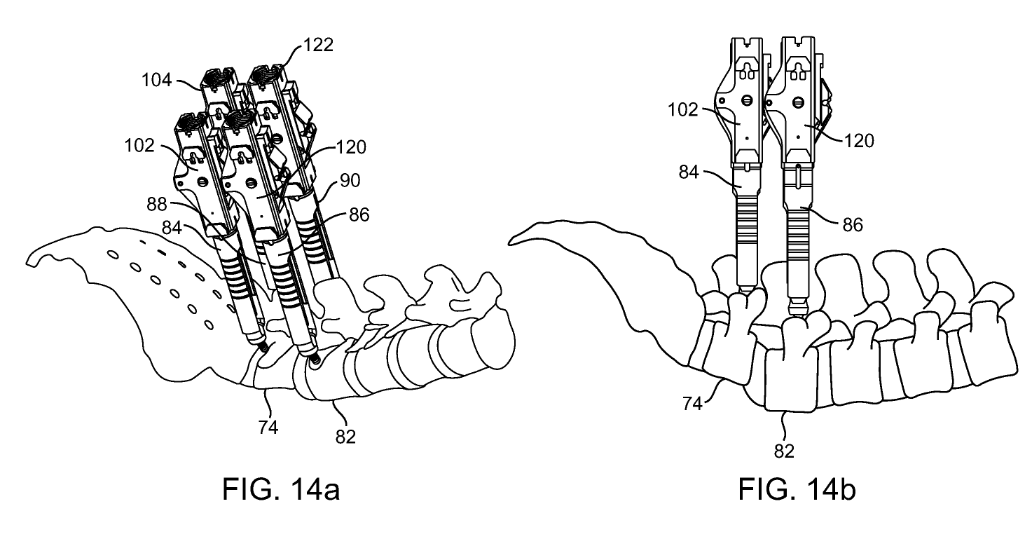

Turning now to FIGS. 14a and 14b, after at least one of the first and third alignment guides 102, 104 are connected to either one or both of the first and third towers 84, 88, a second alignment guide 120 is connected to the proximal end of the second tower 86 and a fourth alignment guide 122 is connected to the proximal end of the fourth tower 90 such that the post 118 and hook 116 of the first and second alignment guides 102, 120 and the post 118 and hook 116 of the third and fourth alignment guides 104, 122 are connected together. Because of the spondylolisthesis, the first vertebral body 74 is shifted posteriorly relative to the second vertebral body 82 and the first and third towers 84, 88 are, therefore, located relatively higher than the second and fourth towers 86, 90 as is clearly visible in FIG. 14b. Therefore, and because the second and fourth alignment guides 120, 122 are hooked to the relatively higher first and third alignment guides 104, 120, the second and fourth alignment guides 120, 122, depending on the degree of spondylolisthesis, will not initially seat onto the second and fourth towers 86, 90 such that the alignment locks 108 of the guides 120, 122 contact the proximal end of the inner sleeve 96 of towers 86, 90. The distance between the alignment lock 108 and the proximal end of the tower is reduced with the spondy reduction tool 10 before the second and fourth alignment guides 120, 122 can be locked to the second and fourth towers 86, 90. In another variation of the present invention, the second and fourth alignment guides 120, 122 will seat onto the second and fourth towers 86, 90 such that the alignment locks 108 contact the proximal end of the inner sleeve 96 and are capable of being locked to towers 86, 90 prior to reduction. This alternative design requires an alignment lock 108 that is longitudinally longer than shown in FIG. 13b such that the threaded distal portion 112 is located closer to the distal end of the alignment guide so that it contacts the tower and can be locked thereto prior to reduction given the spondylolisthesis. In yet another variation, the longer alignment lock 108 just described is used not only to lock the alignment guide to the tower, but also, to reduce the degree of spondylolisthesis via rotation of the alignment lock 108 with a locking tool which pulls the tower and attached vertebral body upwardly (posteriorly) into substantial alignment and reduction.

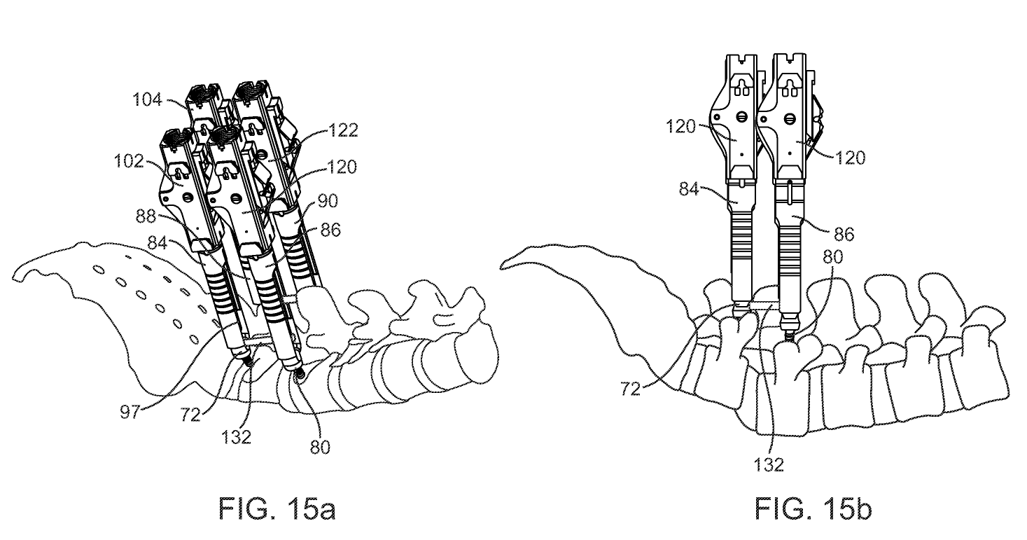

Turning now to FIGS. 15a and 15b, an elongate member or rod 132 is shown with one end connected to the first collar 72 and the second end extending toward the second collar 80. The elongate rod 132 is inserted using a rod inserter and passed in through the longitudinal opening 106 of the alignment guide 102 into the longitudinal opening 92 of the first tower 84 in an orientation parallel to the longitudinal openings 106, 92 and seated in the first collar 72 and then pivoted through the channel 97 to extend toward the second collar 80. A cap (not shown) may then be inserted with a cap inserter to cap the first and second collars 72, 80 and secure the rod thereto. Instruments and methods for inserting and connecting the rod are described in detail in U.S. patent application Ser. No. 11/586,849 entitled “Systems and methods for stabilization of bone structures” filed on Oct. 25, 2006 incorporated herein by reference in its entirety. A second rod (not shown) may be inserted on the opposite side of the vertebrae, that is, inserted in the third construct and secured to the third collar with a cap. Inserting the rods prior to reduction eliminates the need to use a screw locking tool described hereinbelow to lock the polyaxial motion of the screw. In one variation according to the present invention, the rod 132 is not inserted until after the spondylolisthesis of the vertebral bodies is reduced.

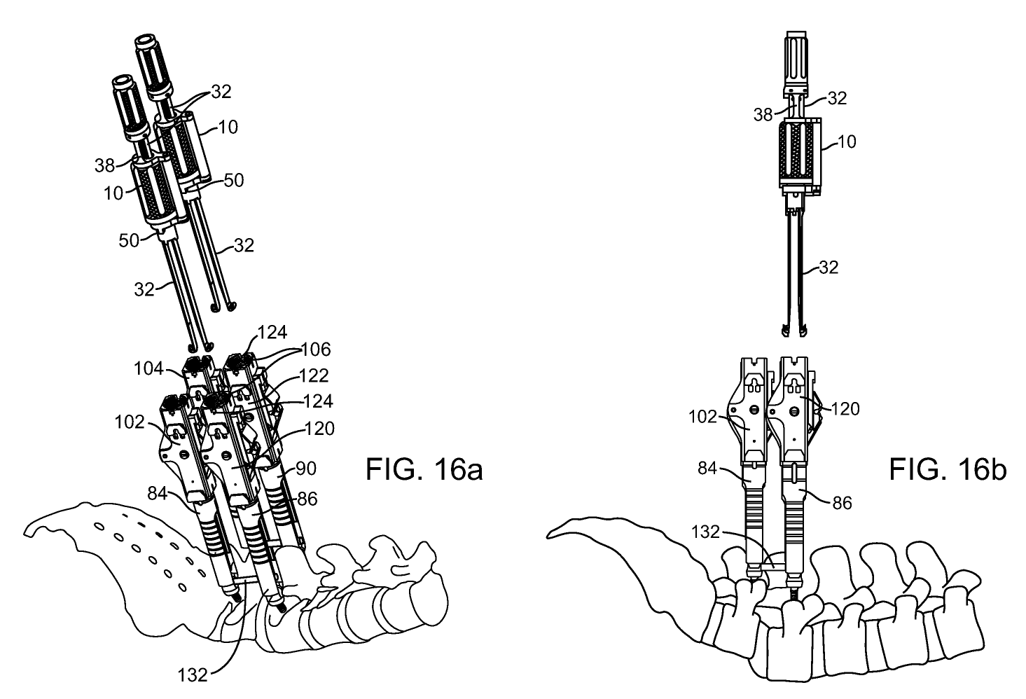

Turning now to FIGS. 16a and 16b, a pair of spondy reduction instruments 10 is introduced and located above the longitudinal openings 106 of the second and fourth alignment guides 120, 122. To insert the spondy reduction tool 10, the user presses together the prongs 32 through the lateral cage windows 38 and inserts the distal end of the spondy reduction tool 10 into the longitudinal opening 106. The locator extensions 50 of the lower cage 20 are inserted into the notches 124 in the top of the alignment guide 120 to align the reduction tool 10.

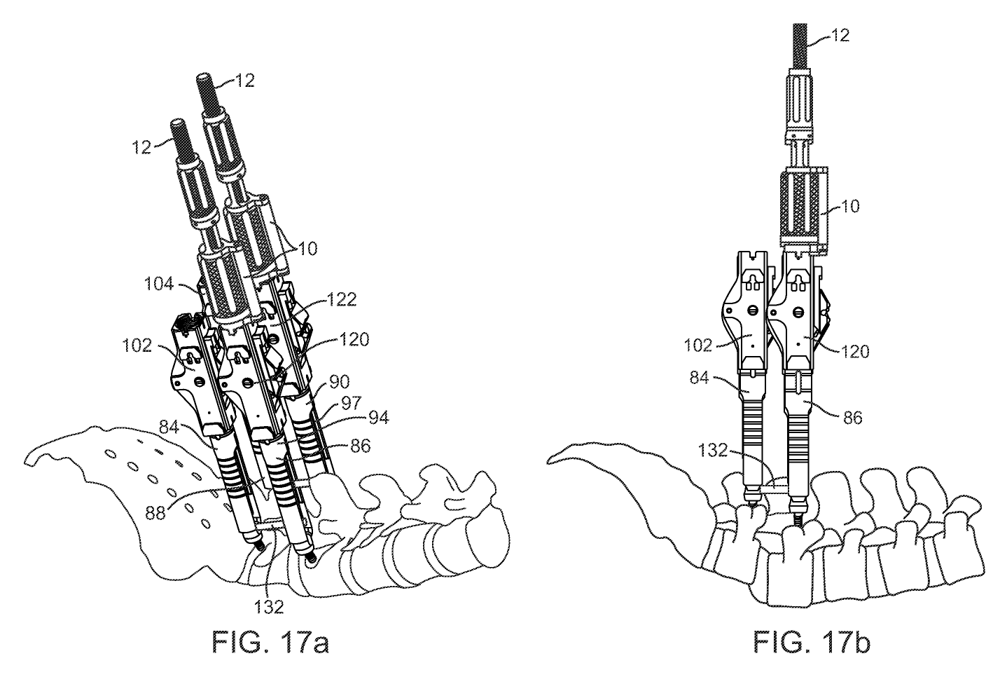

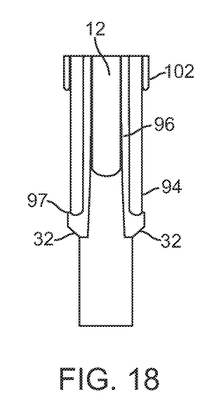

Turning now to FIGS. 17a and 17b, there is shown two spondy reduction tools 10 inserted into the constructs. The inner shaft 12 is inserted into the central bore 26 of spondy reduction tool 10 and passed to expand the prongs 32 until they extend outwardly through the channels 97 and hook onto the outer sleeve 94 at the proximal end of the channel 97 as shown in FIGS. 17a and 17b and in greater detail in FIG. 18.

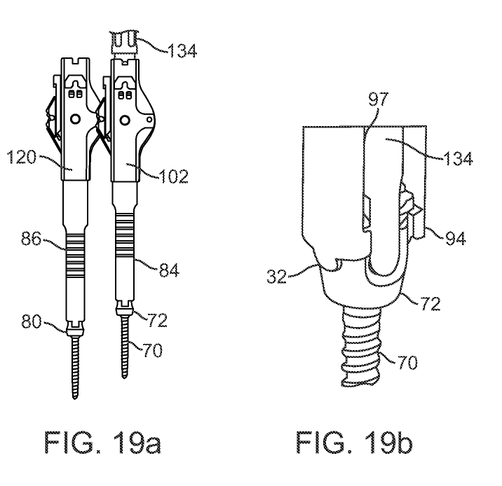

In the variation of the present invention in which the rod 132 is not inserted prior to reducing, a screw locking tool 134 is inserted into each of the first and third alignment guides 102, 104 and passed distally into the first 72 and third collar (not shown) as illustrated in greater detail in FIGS. 19a and 19b. The screw locking tool 134 comprises a shaft portion and a proximal threaded portion. The distal end of the screw locking tool 134 is threadingly advanced into the first collar 72 until a force is exerted sufficient to lock any polyaxial motion that the screw 70 may exhibit during reduction. The screw locking tool 134 assists in stabilizing the construct when large forces are applied during reduction. A second screw locking tool 134 may be employed to lock polyaxial motion of the third collar in the same manner.

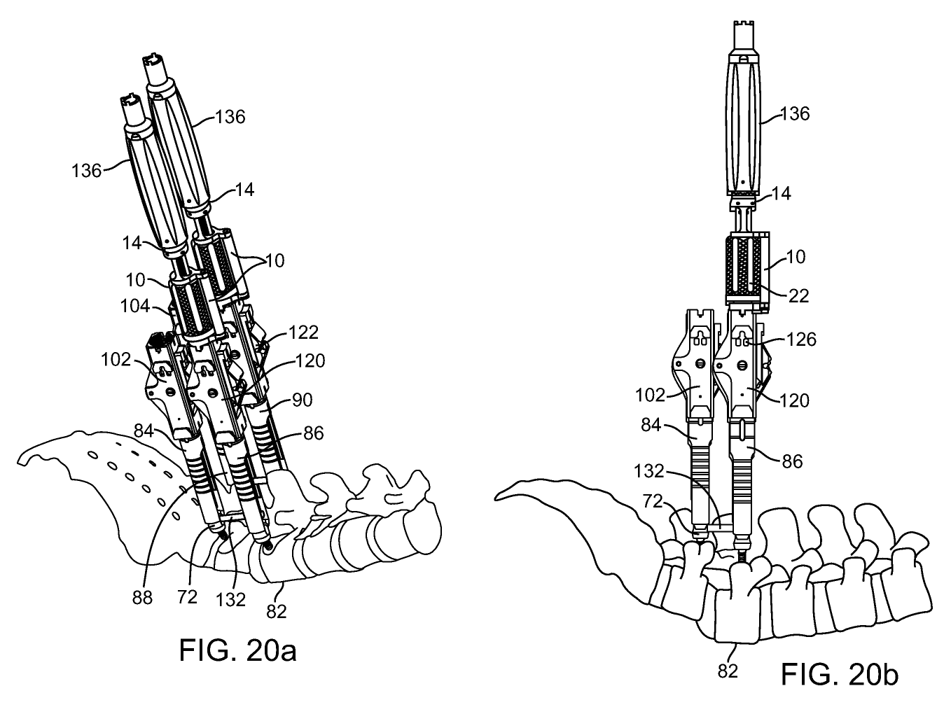

Turning now to FIGS. 20a and 20b, the upper knob 14 of each reduction instrument 10 is rotated to retract the tangs 16 proximally. The user alternates rotating the upper knob 14 of the reduction instrument 10 connected to the second construct and the upper knob 14 of the reduction instrument 10 connected to the fourth construct so as to substantially equally and bilaterally raise and reduce the vertebral bodies so that no unequal stresses are imparted. To assist in the reduction, a locking tool 136 is passed over the upper knob 14 to provide a handle for easily gripping the instrument 10 while turning the upper knob 14. As the upper knob 14 is rotated, the tangs 16 are retracted, thereby, pulling generally upwardly or proximally the tower to which the hooks 34 of the prongs 32 as shown in the drawings or in a general posterior direction relative to the adjacent construct. The tower being connected to the bone screw assembly which is in turn connected to the vertebral body, pulls the second vertebral body 82 upwardly or generally posteriorly to reduce the spondylolisthesis. The upper knob 14 is turned until it is met with resistance. Sufficient reduction of the tower is confirmed by the user looking through the window 126 in the alignment guide. If the tower is visible through the window 126, sufficient reduction is ascertained. If the tower is not visible through the window 126 or is a distance away from the lock 108, the upper knob 14 is rotated to reduce the tower further. When there is sufficient reduction, ascertained through the window and indicated by the tower being proximate to the lock 108, the lower knob 22 is rotated to lock the alignment guide to the tower which is also confirmed by the user by looking through the window 126. Since the extensions 57 of the lower knob 22 are engaged with the notches 128 of the lock 108 in the alignment guide, rotation of the lower knob 22 rotates the lock 108 which threads to the threaded inner surface of the inner sleeve 96 to lock the alignment guide to the tower.

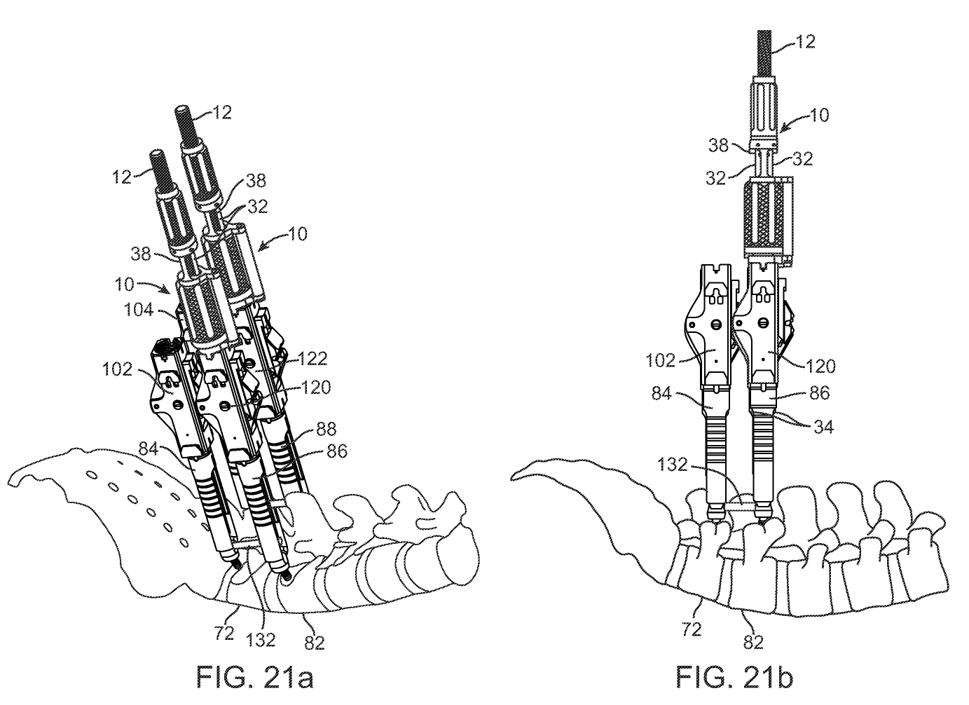

FIGS. 21a and 21b show the reduced vertebral bodies 72, 82. The reduction instrument 10 is removed by first removing the inner shaft 12 and then pressing the prongs 32 through the lateral cage windows 38 to release the hooks 34 from the tower. The reduction instrument is then removed from the construct. A cap inserter (not shown) may then be inserted into the second and fourth constructs to cap the second and fourth collars and secure the rod 132 inside the bone fastener assembly.

In the variation in which a rod 132 has not been previously inserted, it is inserted after reduction, pivoted toward the second collar, capped and locked into the first collar and second collars. A second rod is connected between the third and fourth collars as well in a similar manner. A locking tool 136 is inserted into the alignment guide such that the extensions on the locking tool engage with notches 128 on the lock 108 inside the alignment guide. The locking tool 136 is then rotated to unlock the alignment guide from the tower and the alignment guide is lifted off the tower. This procedure is performed to remove all alignment guides. The same locking tool 136 is inserted into the proximal end and longitudinal opening of the tower such that the extensions on the locking tool engage with the notches 101 in lock 100 of the tower. The locking tool 136 is then rotated to retract the outer sleeve 94 such that the collar engaging prongs 98 disengage from the collar and the tower is removed from the patient. This procedure is performed to remove all towers. The incisions in the patient are then sewn and the procedure is finished.

The disclosed devices or any of their components can be made of any biologically adaptable or compatible materials including PEEK, PEK, PAEK, PEKEKK or other polyetherketones. Materials considered acceptable for biological implantation are well known and include, but are not limited to, stainless steel, titanium, tantalum, combination metallic alloys, various plastics, polymers, resins, ceramics, biologically absorbable materials and the like.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. A spondylolisthesis reduction system for a patient's spine comprising:
a bone anchor implantable into a vertebral body of the patient's spine; the bone anchor having an upper rod receiving portion connected to a lower shank portion;
a guide tube having an upper portion and a lower portion with the lower portion connectable to the bone anchor; the guide tube having a longitudinal opening;
an instrument connectable to the upper portion of the guide tube and configured to move the lower portion of the guide tube relative to the upper portion.

2. The system of claim 1 wherein the instrument includes at least one prong insertable into the longitudinal opening of the guide tube and configured to connect the instrument to the lower portion of the guide tube.

3. The system of claim 2 wherein the lower portion of the guide tube includes at least one sidewall opening formed in the sidewall of the guide tube and the at least one prong is configured to be insertable into the at least one sidewall opening to connect the instrument to the lower portion of the guide tube.

4. The system of claim 1 wherein the instrument is connectable to an adjacent construct to bias movement of the lower portion of the guide tube relative to the upper portion of the guide tube.

5. The system of claim 4 wherein the adjacent construct includes a second bone anchor implantable into a second vertebral body and a second guide tube connectable to the second bone anchor; the second guide tube having a longitudinal opening.

6. The system of claim 5 wherein the second guide tube includes an upper portion and a lower portion with the lower portion connectable to the second bone anchor; the upper portion of the first guide tube being configured to connect to the upper portion of the second guide tube.

7. The system of claim 5 further including an elongated member configured to be delivered through one of the first or second guide tubes and connectable to the first and second bone anchors.

8. A spondylolisthesis reduction system for a patient's spine comprising:
an upper knob; at least one hook connected to the upper knob and configured such that the at least one hook is capable of longitudinal translation relative to the upper knob; the at least one hook configured to be connectable with a tower having a longitudinal opening; the at least one hook configured to longitudinally translate a connected tower along with the longitudinal translation of the at least one hook; the tower being configured to be connectable to a bone fastener assembly implanted in a vertebral body of the patient's spine; wherein translation of the at least one hook, connected tower and vertebral body relative to the upper knob reduces spondylolisthesis of the patient's spine.

9. The system of claim 8 wherein the at least one hook is insertable into the longitudinal opening of the tower and hooked to the tower from the inside of the tower.

10. The system of claim 8 wherein the at least one hook includes a flexible portion capable of deflection.

11. The system of claim 10 further including an upper cage connected to the upper knob, the upper cage having at least one window providing proximal access to the at least one hook for deflecting the flexible portion.

12. The system of claim 11 further including:
a lower cage connected to the upper cage;
a lower knob connected between the upper cage and the lower cage;
wherein the tower includes a removable alignment guide having a lock and the lower knob is configured to engage with the lock of an alignment guide to lock the alignment guide and tower together.

13. The system of claim 10 wherein the at least one hook is insertable into the longitudinal opening of the tower and hooked to the tower from the inside of the tower; the system further including a removable inner shaft insertable into the longitudinal channel for expanding the flexible portion of the at least one hook to hook the flexible portion to the tower.

14. The system of claim 8 further including an elongated member deliverable through the longitudinal opening and connectable to the bone fastener assembly.

15. The system of claim 8 wherein the upper knob is configured to longitudinally translate the at least one hook relative to the upper knob wherein the system is configured such that the upper knob resides outside the patient's body when the at least one hook is connected to a tower connected to the bone fastener assembly inside the patient's body.

16. A method for reducing spondylolisthesis in a patient's spine, comprising the steps of:
providing at least a first bone anchor implanted in a first vertebral body;
providing at least a second bone anchor implanted in a second vertebral body;
providing a first tower;
connecting a first tower to a first bone anchor;
providing a second tower having at least one longitudinal opening, an upper portion and a lower portion;
connecting a second tower to a second bone anchor;
connecting the first tower to the upper portion of the second tower;
providing an instrument comprising an upper portion connected to at least one prong;
inserting the instrument into the second tower;
connecting the at least one prong to the second tower; and
moving the prong proximally to move the second tower, the connected second bone anchor, and the connected second vertebral body proximally to align the first and second vertebral bodies.

17. The method of claim 16 further including the steps of:
removing the instrument;
providing an elongated member;
inserting the elongated member into one of the first or second towers;
connecting one end of the elongated member to the first bone anchor; and
connecting the other end of the elongated member to the second bone anchor.

18. The method of claim 16 further including the steps of:
providing a first tower having an upper portion and a lower portion;
wherein the step of connecting the first tower to the upper portion of the second tower includes connecting the upper portion of the first tower to the upper portion of the second tower; and
wherein the step of moving the prong proximally to move the second tower includes moving the prong proximally to move the lower portion of the second tower proximally relative to the upper portion of the second tower.

19. The method of claim 16 wherein the step of moving the prong proximally includes rotating the upper portion of the instrument to move the prong proximally relative to the upper portion of the instrument.

20. The method of claim 16 further including the step of:
providing a prong expander; and
inserting the prong expander into the instrument to move the at least one prong into the at least one longitudinal opening.

21. The method of claim 16 wherein the step of connecting the at least one prong to the second tower includes hooking the at least one prong into the at least one longitudinal opening of the second tower.
